(19) United States
(12) Patent Application Publication
(10) Pub. No.: US 2020/0187855 A1
(43) Pub. Date: Jun. 18, 2020

(54) LIMB DETECTION DEVICE

(71) Applicant: METAL INDUSTRIES RESEARCH & DEVELOPMENT CENTRE, Kaohsiung City 811 (TW)

(72) Inventors: Ming-Hui Chen, Kaohsiung City (TW); Bing-Feng Huang, Kaohsiung City (TW); Hsiang-Hsiang Choo, Taipei City (TW); Tzyy-Ker Sue, Kaohsiung City (TW)

(21) Appl. No.: 16/220,126

(22) Filed: Dec. 14, 2018

(51) Int. Cl. A61B 5/00; A61B 8/00

(52) U.S. Cl. CPC A61B 5/4893; A61B 17/58; A61B 8/4227

(57) ABSTRACT

A device for limb detection includes a circular guide means and a carrier. The carrier is provided to carry an image detector and the circular guide means includes a slide mechanism integrated to the carrier. The slide mechanism and the carrier can move back and forth around a horizontal axis passing through the circular guide means such that they are beneficial for the image detector to detect the limb.

### FIELD OF THE INVENTION

This invention generally relates to a device used for detecting a limb, and more particularly to a device used for identifying and locating nerves and blood vessels in the limb.

### BACKGROUND OF THE INVENTION

In clinical practice, implanting a locking plate into patient's broken limb by minimally invasive surgery is usually adopted for orthopedic trauma. The locking plate avoiding soft tissue, such as nerves and blood vessels, is necessary during implantation because the locking plate compressing the nerves and blood vessels may induce postoperative pain or paralysis.

However, a doctor only can predict possible locations of the nerves and blood vessels in the passing path of the locking plate according to human anatomy. And even referring to X-ray image of the patient, risks of nerve and blood vessel compression result from the blocking plate still exist during the surgery.

### SUMMARY

One object of the present invention is to provide a limb detection device which can carry and move an image detector on a limb stably to search nerves and blood vessels in the limb for identifying and locating.

The limb detection device of the present invention comprise a circular guide means and a carrier. The circular guide means includes a slide mechanism which can slide back and forth around a horizontal axis passing through the circular guide means. The carrier having a body, a carry rod and a shaft is integrated with the slide mechanism by the body. The carry rod having a drive portion and a carry portion is movably coupled to the body by the drive portion such that the carry rod can move toward or away from the horizontal axis selectively. The carry portion is movably coupled to the drive portion and is used to carry the image detector. The shaft penetrates through the drive portion along a direction of a vertical axis crossing the horizontal axis and can be rotated in the drive portion selectively. Further, the shaft is integrated with the carry portion by a first end such that the carry portion, the image detector and the shaft can be rotated back and forth around the vertical axis.

In the limb detection device of the present invention, the slide mechanism and the carrier are provided to move the image detector to touch the limb for identifying and locating the nerves and blood vessels in the limb. As a result, nerves and blood vessel compression caused by an implant (e.g. locking plate) implanted in the patient and postoperative pain or paralysis are preventable.

### DESCRIPTION OF THE DRAWINGS

FIG. 1 is a perspective assembly diagram illustrating a limb detection device in accordance with an embodiment of the present invention.

FIG. 2 is a perspective assembly diagram illustrating the limb detection device in accordance with an embodiment of the present invention.

FIG. 3 is a perspective exploded diagram illustrating the limb detection device in accordance with an embodiment of the present invention.

FIG. 4 is a partial perspective exploded diagram illustrating the limb detection device in accordance with an embodiment of the present invention.

FIG. 5 is a cross-sectional diagram illustrating the limb detection device in accordance with an embodiment of the present invention.

FIG. 6 is a perspective assembly diagram illustrating a carrier of the limb detection device in accordance with an embodiment of the present invention.

FIG. 7 is a perspective exploded diagram illustrating the carrier of the limb detection device in accordance with an embodiment of the present invention.

FIG. 8 is a cross-sectional perspective diagram illustrating the carrier of the limb detection device in accordance with an embodiment of the present invention.

### DETAILED DESCRIPTION OF THE INVENTION

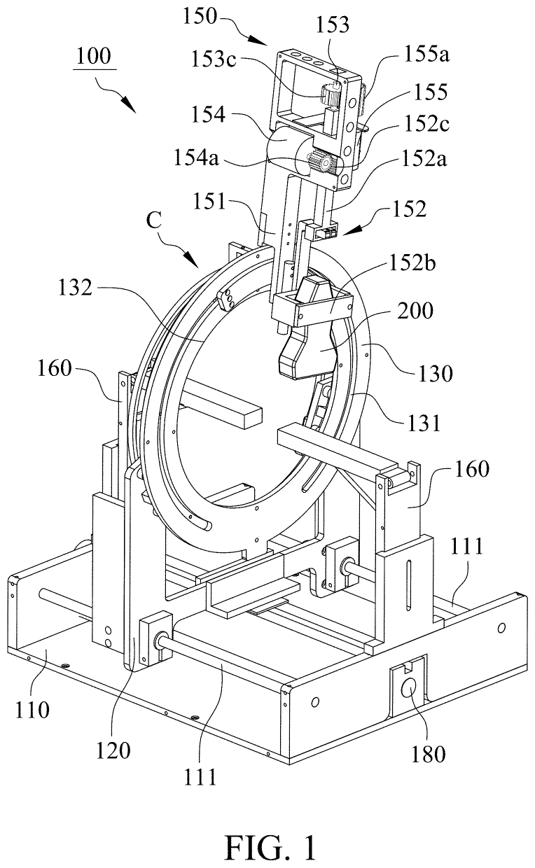

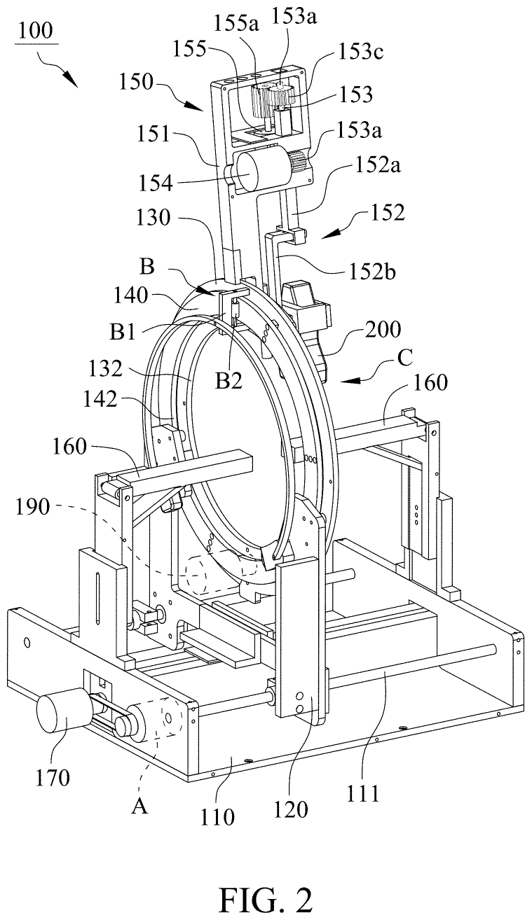

With reference to FIGS. 1 and 2, a limb detection device 100 of the present invention is provided to carry an image detector 200 for identifying and locating nerves and blood vessels in a limb (not shown). The image detector 200 may be an ultrasonic detector.

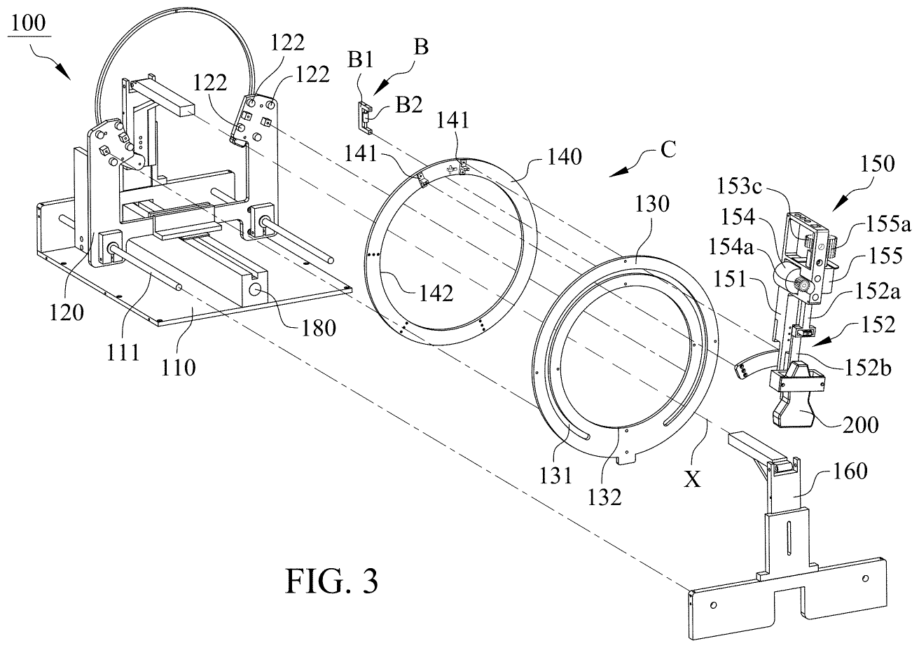

With reference to FIGS. 1 and 3, the limb detection device 100 includes a foundation 110, a slide base 120, a circular guide means C, a carrier 150 and at least one bracket 160. The circular guide means C in this embodiment includes a limit plate 130 and a slide mechanism 140. The bracket 160 is arranged on the foundation 110 and provided to support the limb. The foundation 110 includes at least one track 111, the slide base 120 is movably mounted on the track 111 and can be moved back and forth along the track 111. The limit plate 130 is coupled to the slide base 120 by, but not limited to, a screw such that they can move together. There is an arcing limit slot 131 penetrating through the limit plate 130, and in this embodiment, there is further a first accommodation opening 132 on the limit plate 130 for accommodating the limb.

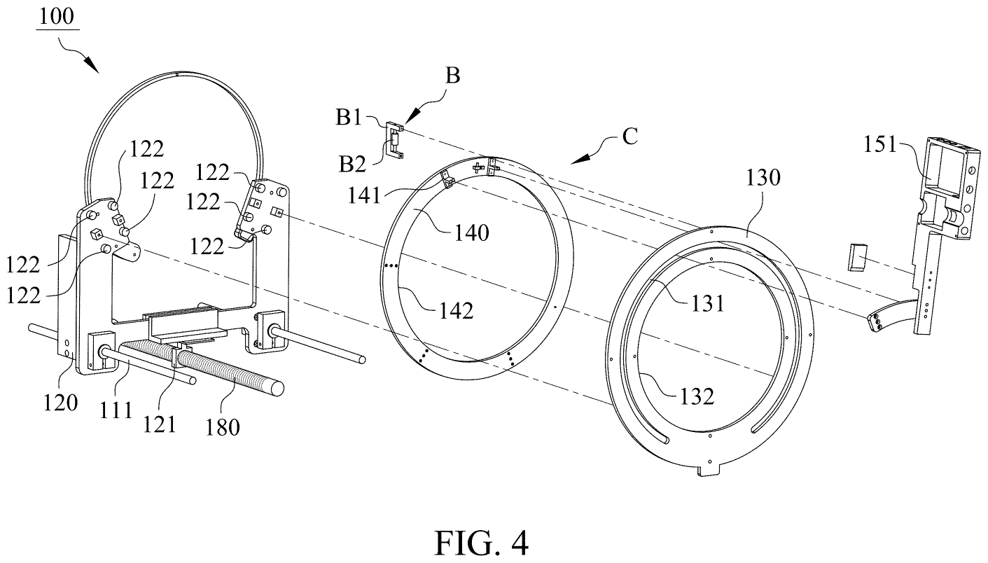

With reference to FIGS. 3 and 4, the slide mechanism 140 and the carrier 150 are arranged on both sides of the limit plate 130 respectively, in other words, the slide mechanism 140 is placed on one side of the limit plate 130 and the carrier 150 is placed on the other side of the limit plate 130 such that the limit plate 130 is located between the slide mechanism 140 and the carrier 150. The slide mechanism 140 can slide back and forth around a horizontal axis X passing through the circular guide means C and has a second accommodation opening 142 used to accommodate the limb. The carrier 150 is provided to carry the image detector 200 and direct the image detector 200 toward the limb.

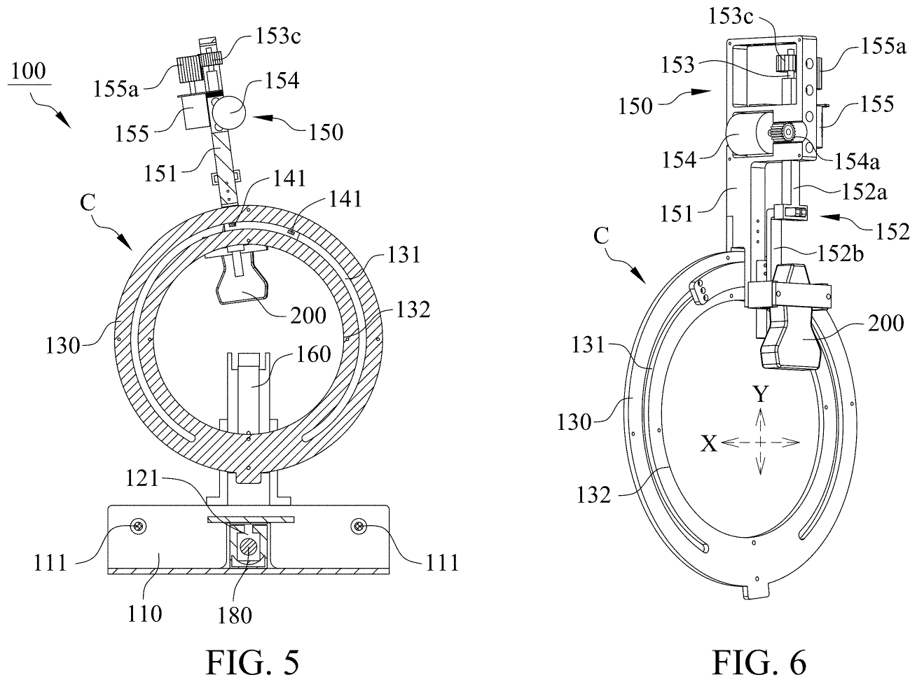

With reference to FIGS. 3, 4 and 5, the slide mechanism 140 includes at least one limiter 141, and in this embodiment, the slide mechanism 140 has two limiters 141. The limiter 141 in other embodiments may be an arcuate rib having a radian substantially equal to that of the arcing limit slot 131. The limiter 141 is inserted in the arcing limit slot 131 and can be moved in the arcing limit slot 131. By the limiter 141 confined in the arcing limit slot 131, the slide mechanism 140 is integrated with the carrier 150 and can slide back and forth around the horizontal axis X passing through the limit plate 130. Preferably, the slide base 120 includes a plurality of rollers 122 which are provided to support the slide mechanism 140 for stable rotation. The carrier 150 can move back and forth around the horizontal axis X with the slide mechanism 140 in rotation.

With reference to FIGS. 2 and 4, the limb detection device 100 further includes a roller set B having a support B1 and at least one roller B2. The support B1 is connected to the limit plate 130, the roller B2 is mounted on the support B1 and contacts the slide mechanism 140. The slide mechanism 140 is located between the roller B2 and the limit plate 130, and the roller set B is provided for stable rotation of the slide mechanism 140 by the roller B2 which contacts the rotated slide mechanism 140.

With reference to FIG. 6, the carrier 150 includes a body 151 and a carry rod 152 and is integrated with the slide mechanism 140 via the body 151. The carry rod 152 is provided to carry the image detector 200 and is connected to the body 151 movably, further, the carry rod 152 can move toward or away from the horizontal axis X selectively.

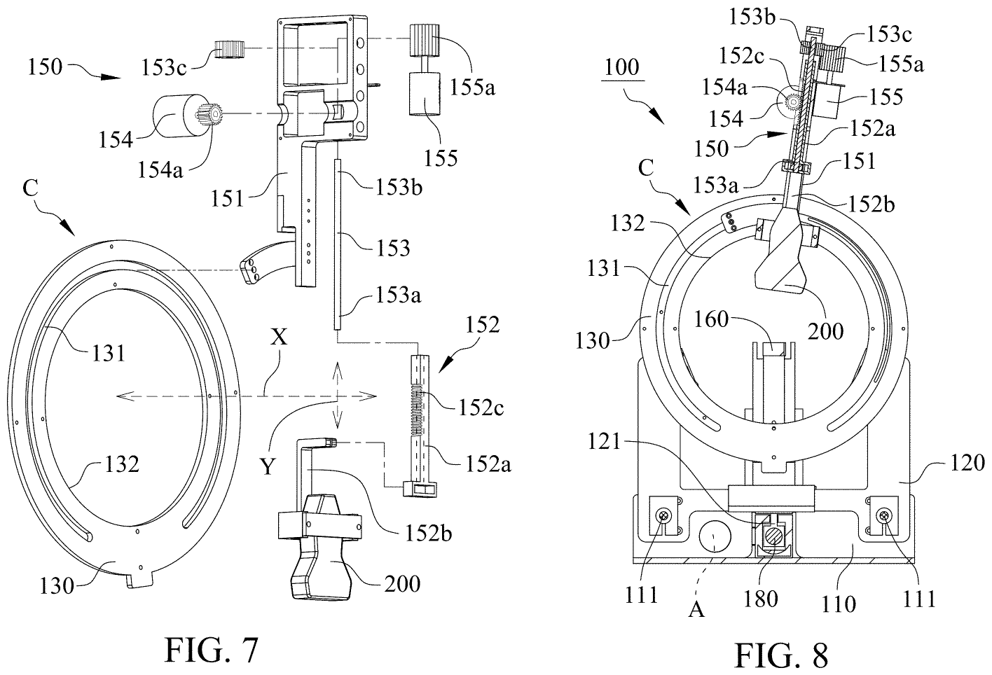

With reference to FIGS. 7 and 8, in this embodiment, the carrier 150 further includes a shaft 153 and the carry rod 152 has a drive portion 152a and a carry portion 152b. The image detector 200 is mounted on the carry portion 152b which is movably coupled to the drive portion 152a. The carry rod 152 is connected to the body 151 via the drive portion 152a such that the image detector 200 can be selectively moved toward or away from the horizontal axis X. The shaft 153 penetrates through the drive portion 152a along a direction of a vertical axis Y which crosses the horizontal axis X and can rotate in the drive portion 152a selectively. The shaft 153 is integrated to the carry portion 152b by its first end 153a so the carry portion 152b, the image detector 200 and the shaft 153 can rotate back and forth around the vertical axis Y.

With reference to FIGS. 7 and 8, the carrier 150 further includes a first motor 154 that is used to move the carry rod 152 back and forth along the direction of the vertical axis Y so that the image detector 200 can be moved toward or away from the horizontal axis X selectively. In this embodiment, a first contact element 154a and a second contact element 152c are disposed on the first motor 154 and the drive portion 152a respectively and in contact with each other, consequently, the first motor 154 can move the carry rod 152 back and forth. The first contact element 154a and the second contact element 152c may be a gear and a rack engaged with each other respectively.

With reference to FIGS. 7 and 8, the carrier 150 further includes a second motor 155. A third contact element 155a and a fourth contact element 153c are respectively disposed on the second motor 155 and a second end 153b of the shaft 153 and in contact with each other. Thus, the second motor 155 can drive the shaft 153 in rotation and further drive the carry portion 152b and the image detector 200 rotate together. In this embodiment, the third contact element 155a and the fourth contact element 153c are respectively a gear and a rack engaged with each other.

With reference to FIGS. 2 and 4, the limb detection device 100 in this embodiment further includes a third motor 170, a drive rod 180 and a fourth motor 190. The slide base 120 is pivotally connected to the drive rod 180 by a picot portion 121, the third motor 170 is used to drive the drive rod 180 in rotation so as to move the slide base 120 back and forth along the track 111, and the fourth motor 190 is provided to drive the slide mechanism 140 sliding back and forth.

With reference to FIGS. 2 and 4, the limb detection device 100 preferably further includes at least one encoder A provided to record the drive data of the third motor 170 for displacement conversion by using a computation system. In this embodiment, each of the first motor 154, the second motor 155, the third motor 170 and the fourth motor 190 is integrated with an encoder (not shown) which is also used to record the drive data of the motor for a computation system performing displacement conversion.

With reference to FIGS. 1 and 2, when a limb (not shown) is placed on the bracket 160, the limb is accommodated in the first accommodation opening 132 of the limit plate 130 and the second accommodation opening 142 of the slide mechanism 140 simultaneously. The image detector 200 is firstly moved to a position above a determined region of surgery by hand or by using the third motor 170 which can move the slide base 120 to allow the limit plate 130, the slide mechanism 140 and the carrier 150 to move to the position above the determined region of surgery, next, the image detector 200 is aligned with the determined region by hand or by using the fourth motor 190 to rotate the slide mechanism 140, and then, the image detector 200 is moved to touch the determined region on the limb by hand or by using the first motor 154 to move the shaft 152 of the carrier 150, furthermore, the image detector 200 can be moved slightly on the determined region by hand or by using the second motor 155 to rotate the shaft 153 such that the image detector 200 can search nerves and blood vessels under the determined region. Additionally, in order to locate the nerves and blood vessels, the encoders A may be provided to record the drive data of the motors for displacement conversion processed in a computation system.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A limb detection device used to carry an image detector, comprising:
a circular guide means including a slide mechanism, the slide mechanism is able to slide back and forth around a horizontal axis passing through the circular guide means; and
a carrier including a body, a carry rod and a shaft, the carrier is integrated with the slide mechanism by the body, the carry rod includes a drive portion and a carry portion and is movably coupled to the body by the drive portion so as to be able to be moved toward or away from the horizontal axis selectively, the carry portion is movably coupled to the drive portion and is used to carry the image detector, the shaft penetrates through the drive portion along a direction of a vertical axis crossing the horizontal axis and is able to be rotated in the drive portion selectively, the shaft is integrated with the carry portion by a first end such that the carry portion, the image detector and the shaft are able to be rotated back and forth around the vertical axis.

2. The limb detection device in accordance with claim 1, wherein the circular guide means further includes a limit plate, and there is an arcing limit slot penetrating through the limit plate, the slide mechanism is disposed on one side of the limit plate and includes at least one limiter, the limiter is inserted in the arcing limit slot and able to be moved in the arcing limit slot selectively, and the slide mechanism is able to slide back and forth around a horizontal axis passing through the limit plate because of the limiter confined in the arcing limit slot.

3. The limb detection device in accordance with claim 2, wherein the carrier is disposed on the other side of the limit plate such that the limit plate is located between the slide mechanism and the carrier, and the slide mechanism is integrated with the carrier by the limiter such that the carrier is able to be moved back and forth around the horizontal axis.

4. The limb detection device in accordance with claim 2 further comprising a foundation and a slide base, wherein the foundation includes at least one track, the slide base is movably mounted on the track and is able to be moved back and forth along the track, the limit plate is coupled to the slide base such that the limit plate is able to be moved with the slide base.

5. The limb detection device in accordance with claim 1, wherein the carrier further includes a first motor, a first contact element is disposed on the first motor and contacts a second contact element disposed on the drive portion such that the first motor is able to move the carry rod back and forth.

6. The limb detection device in accordance with claim 1, wherein the carrier further includes a second motor, a third contact element is disposed on the second motor and contacts a fourth contact element disposed on a second end of the shaft such that the second motor is able to drive the shaft in rotation and further drive the carry portion and the image detector rotate together.

7. The limb detection device in accordance with claim 2, wherein the limit plate has a first accommodation opening and the slide mechanism has a second accommodation opening, the first and second accommodation openings are provided to accommodate a limb.

8. The limb detection device in accordance with claim 4 further comprising at least one bracket disposed on the foundation, wherein the bracket is provided to support a limb.

9. The limb detection device in accordance with claim 4 further comprising a third motor and a drive rod, wherein the slide base is pivotally connected to the drive rod by a pivot portion, and the third motor is used to drive the drive rod in rotation to move the slide base back and forth along the track.

10. The limb detection device in accordance with claim 1 further comprising a fourth motor used to drive the slide mechanism slide back and forth.

11. The limb detection device in accordance with claim 5 further comprising at least one encoder, wherein the encoder is used to record a drive data of the first motor for displacement conversion performed by a computation system.

12. The limb detection device in accordance with claim 6 further comprising at least one encoder, wherein the encoder is used to record a drive data of the second motor for displacement conversion performed by a computation system.

13. The limb detection device in accordance with claim 9 further comprising at least one encoder, wherein the encoder is used to record a drive data of the third motor for displacement conversion performed by a computation system.

14. The limb detection device in accordance with claim 10 further comprising at least one encoder, wherein the encoder is used to record a drive data of the fourth motor for displacement conversion performed by a computation system.

15. The limb detection device in accordance with claim 4, wherein the slide base includes a plurality of rollers used for supporting the slide mechanism.

16. The limb detection device in accordance with claim 2 further comprising a roller set having a support and at least one roller, wherein the support is coupled to the limit plate, the roller is disposed on the support and contacts the slide mechanism, the slide mechanism is located between the roller and the limit plate.
